(19) United States
(12) Patent Application Publication
(10) Pub. No.: US 2007/0141036 A1
(43) Pub. Date: Jun. 21, 2007

(54) Composition and procedure for tissue creation, regeneration and repair by a cell-bearing biological implant enriched with platelet concentrate and supplements

(76) Inventors: Alberto Gorrochategui Barrueta, Bilbao (ES); Josu Simon Elizundia, Bilbao (ES)

Correspondence Address: GALLAGHER & DAWSEY CO., L.P.A., P.O. BOX 785, COLUMBUS, OH, 43216, US

(21) Appl. No.: 11/704,784

(22) Filed: Feb. 9, 2007

Related U.S. Application Data
(63) Continuation-in-part of application No. 10/475,866, filed on Oct. 24, 2003.

(51) Int. Cl. A61K 35/14

(52) U.S. Cl. 424/93.7

(57) ABSTRACT

A composition and method for enhancing tissue growth, regeneration, and repair includes a Biological Glue formed by extraction of an Extremely Platelet Rich Plasma (EPRP) derived from whole blood, and subsequent activation and clotting. The Biological Glue may be utilized alone to fill defects or may be used as an adhesive agent for other biological and non-biological materials. These materials may include processed thrombus derived from the activation of EPRP. Additionally, the Extremely Platelet Rich Plasma may be impregnated with directly harvested or cultured cells, including stem cells, or other materials, prior to activation, to form a Biological Implant that may be implanted in vivo. A Platelet Factor Enriched Serum (PFS) derived from the activation of the Extremely Platelet Rich Plasma (EPRP) may be added to the cell cultures in preparation of a Biological Implant, in order to provide additional growth factors that speed the development of the cell cultures.

### PRIORITY CLAIM

This application is a continuation-in-part of an application filed in the United States Patent and Trademark Office under 35 U.S.C. 371 on Oct. 24, 2003 and given Ser. No. 10/475,866. This application claims priority to PCT application number PCT/ES02/00007, filed on Jan. 9, 2002, and published as International Publication number WO 03/057865 A1 on Jul. 17, 2003.

### BACKGROUND OF THE INVENTION

Techniques of in vitro cell culture have been well known for many years. Recently, the proliferation of research in stem cell applications has led to increased interest in maintaining and using these omnipotent cells.

The in vitro research has highlighted numerous problems in practical application. Cultured cells are often difficult to grow and maintain in vitro, and can be difficult to apply to physiologic settings. It can be difficult to cause cultured cells to adhere to physiological sites, and difficult to stimulate these cells to grow after in vivo implantation.

Platelets are well known to affect wound healing. Platelet extracts show a high mitogenous activity and have a known scarring effect. Numerous products from platelet degranulation are known to affect cell growth; including serotonin, catecholamines, ATP and ADP, and calcium ions from dense granules; and albumin, beta-thromboglobulin, osteonectin, osteocalcin, platelet activation factor 4, platelet derived endothelial growth factor, and endothelial growth factor from alpha granules. Additional components include alpha plasmin inhibiting factor, fibrinogen, proacelerin, fibronectin, connective tissue activation peptide III, transforming beta factor, insulin type growth factor, high molecular mass quininogen, von Willebrand factor, thromospondin, phsopholipid, C1-sterease inhibitor, hepatocyte growth factor, and other platelet derived factors.

Some attempts have been made in the prior art to utilize platelets and platelet derived factors to promote healing. A remote antecedent to the instant invention is International Patent Application WO 90/07931 ('931) to Curatech, Inc., related to U.S. Pat. Nos. 4,957,742 and 5,178,883, relating to recovery factors for pilous follicles using platelet growth factors. The '931 method employs platelets, activated by thrombin, which release platelet derived growth and angiogenesis factors. These factors are combined with a microcrystalline collagen carrier to form a salve that may be applied to wounds to promote healing.

### SUMMARY OF INVENTION

In its most general configuration, the present invention advances the state of the art with a variety of new capabilities and overcomes many of the shortcomings of prior devices in new and novel ways. In its most general sense, the present invention overcomes the shortcomings and limitations of the prior art in any of a number of generally effective configurations. The instant invention demonstrates such capabilities and overcomes many of the shortcomings of prior methods in new and novel ways.

In one configuration, the present invention relates to a composition and method for tissue creation, regeneration and repair by, in some embodiments, a cell-free Biological Glue or a cell-bearing Biological Implant enriched with platelet concentrate and supplements. The method is described in schematic form in FIGS. 1 and 2. Blood is initially fractionated into a Blood Cell (BC) component and Platelet Rich Plasma (PRP) component. The Blood Cell (BC) component is removed from further use in the method and may be utilized in a myriad of other applications, as would be well known to one skilled in the art. The Platelet Rich Plasma (PRP) component is further fractionated into an Extremely Platelet Rich Plasma Component (EPRP) and a Platelet Poor Plasma component (PPP). The Platelet Poor Plasma (PPP) component is removed from further use in the method and may be utilized in a myriad of other applications, as would be well known to one skilled in the art.

As seen in FIG. 1, the Extremely Platelet Rich Plasma (EPRP) may, in one embodiment, be activated with calcium, as would be know to one skilled in the art, forming a transitory stage of Biological Glue. The Extremely Platelet Rich Plasma (EPRP) would then clot, allowing release of the platelet derived factors (PDFs), including Platelet Derived Growth Factors (PDGFs) and other intra-platelet compounds. Following retraction of the clot, the clot may be removed, leaving cell-free Platelet Factor Enriched Serum (PFS) containing the PDFs and other compounds in the liquid portion. The Platelet Factor Enriched Serum (PFS) may be used to enhance the growth of cell cultures applicable to the method of the instant invention. The clot may be further processed, as will be described, for additional applications, including Biological Filler. By way of example, the clot may be freeze-dried and then ground, to provide a biologically based source of Biological Filler material.

Alternatively, as will be described in the experimental applications below, the transitory stage of the Biological Glue may be used as a form of glue or paint to cause adhesion of cells derived from pre-existing cell cultures, or other materials, including both organic and inorganic materials, to a biological surface. By way of example and not limitation, the Biological Glue may be used with other natural or artificial implant material to enhance adhesion. The Biological Glue favors the healing process due to the platelet derived factors present in the Biological Glue, and which may act on the cells on a wound, inducing cell multiplication, differentiation, or chemotaxis. The Biological Glue may be sufficient in volume to fill certain small tissue defects. Platelets are a primary source of growth factor present in the Biological Glue.

As illustrated in FIGS. 2-4, the Extremely Platelet Rich Plasma (EPRP) may be mixed, and may further be cultured with, cells from pre-existing cell cultures or other materials, including both organic and inorganic materials, to form a matrix in which the cultured cells or other materials are substantially uniformly dispersed throughout the Extremely Platelet Rich Plasma (EPRP.) Upon activation, a clot forms which acts as a biological scaffold, that is, it contains cultured cells or other materials substantially disposed throughout the clot. The clot thus forms a Biological Implant that may be implanted as a space occupying mass into a biological space. This Biological Implant may fill voids, and the cultured cells that it may contain may grow within the implant to regenerate tissue.

The culture medium which nourishes the different cell lines includes doses of various supplements in order to increase the number of cells obtained in the shortest possible period of time. These supplements are mainly nucleosides, hormones, cytosines, amino acids and vitamins, although mineral salts, lipids and other compounds are included.

Additionally, the Platelet Factor Enriched Serum (PFS) added to the culture medium in which these high doses of supplements are added provide the platelet derived factors (PDFs) required to optimize the establishment, maintenance, and extension of proliferation.

These variations, modifications, alternatives, and alterations of the various preferred embodiments, arrangements, and configurations may be used alone or in combination with one another as will become more readily apparent to those with skill in the art with reference to the following detailed description of the preferred embodiments and the accompanying figures and drawings.

### BRIEF DESCRIPTION OF THE DRAWINGS

Without limiting the scope of the present invention as claimed below and referring now to the drawings and figures:

FIG. 1 is a flow chart of the process of preparing Biological Glue, Platelet Factor Enriched Serum (PFS), and Biological Filler, wherein Biological Glue may be introduced in vivo alone, or used to adhere Biological Filler, or used as an adhesive agent for biological or non-biological material placed at an in vivo site;

FIG. 2 is a flow chart of the process of preparing a Biological Implant, wherein harvested cells are mixed with Extremely Platelet Rich Plasma (EPRP) prior to activation and creation of the Biological Implant, thereby dispersing the cells throughout the matrix of the Biological Implant;

FIG. 3 is a flow chart of the process of preparing a Biological Implant wherein harvested cells are cultured in Enhanced Basic Cell Culture Media (EBM) and Platelet Factor Enriched Serum (PFS) prior to mixture with Extremely Platelet Rich Plasma (EPRP) and subsequent activation and creation of the Biological Implant, thereby dispersing the cultured cells, culture media, and Platelet Derived Factors (PDFs) derived from the Extremely Platelet Rich Plasma (EPRP) throughout the matrix of the Biological Implant;

FIG. 4 is a flow chart of the process of preparing a Biological Implant wherein harvested cells are cultured in Enhanced Basic Cell Culture Media (EBM) enriched with Specialized Enhanced Basic Cell Culture Media (SBM) and Platelet Factor Enriched Serum (PFS) prior to mixture with Extremely Platelet Rich Plasma (EPRP) and subsequent activation and creation of the Biological Implant, thereby dispersing the cultured cells, culture media, and Platelet Derived Factors (PDFs) derived from the Extremely Platelet Rich Plasma (EPRP) throughout the matrix of the Biological Implant;

FIG. 5 is a flow chart of the process of preparing a Biological Implant wherein biological or non-biological material, or both, is mixed with Extremely Platelet Rich Plasma (EPRP) prior to activation, dispersing the biological, non-biological, or combined material throughout the matrix of the Biological Implant;

FIG. 6 is a cross-sectional diagram of a tissue defect showing an open defect in a connective tissue matrix surrounded by tissue cells;

FIG. 7 is a cross sectional diagram of the tissue defect of FIG. 6 after having the defect filled with Biological Glue of the instant invention;

FIG. 8 is a cross sectional diagram of the tissue defect of FIG. 6 after having a biological or non-biological material, such as by way of example and not limitation, a prosthetic implant, adhered to the defect filled with Biological Glue;

FIG. 9 is a cross sectional diagram of the tissue defect of FIG. 6 after having the defect filled with a Biological Implant formed by mixing cells, harvested or cultured; stem or committed, in the Extremely Platelet Rich Plasma (EPRP) prior to activation;

FIG. 10 is a cross sectional diagram of the tissue defect of FIG. 6 after having the defect filled with a Biological Implant formed by mixing non-biological material in the Extremely Platelet Rich Plasma (EPRP) prior to activation;

FIG. 11 is a cross sectional diagram of the tissue defect of FIG. 6 after having the defect filled with a Biological Implant formed by mixing Biological Filler in the Extremely Platelet Rich Plasma (EPRP) prior to activation;

FIG. 12 is a cross sectional diagram of the tissue defect of FIG. 6 after having the defect filled with a Biological Implant formed by mixing Biological Filler and cells, harvested or cultured; stem or committed, in the Extremely Platelet Rich Plasma (EPRP) prior to activation;

FIG. 13 is a cross-sectional diagram of an interstitial defect presenting as a potential space (shown expanded) between tissue layers;

FIG. 14 is a cross-sectional diagram of the interstitial defect of FIG. 13 showing the interstitial defect filled with Biological Glue;

FIG. 15 is a cross-sectional diagram of the interstitial defect of FIG. 13 showing the interstitial defect filled with a Biological Implant formed by mixing cells, harvested or cultured; stem or committed, in the Extremely Platelet Rich Plasma (EPRP) prior to activation;

FIG. 16 is a cross-sectional diagram of the interstitial defect of FIG. 13 showing the interstitial defect filled with a Biological Implant formed by mixing Biological Filler in the Extremely Platelet Rich Plasma (EPRP) prior to activation; and

FIG. 17 is a cross-sectional diagram of the interstitial defect of FIG. 13 showing the interstitial defect filled with a Biological Implant formed by mixing Biological Filler and cells, harvested or cultured; stem or committed, in the Extremely Platelet Rich Plasma (EPRP) prior to activation.

### DETAILED DESCRIPTION OF THE INVENTION

The method and materials of biological tissue repair of the instant invention enables a significant advance in the state of the art. The preferred embodiments of the method and materials accomplish this by new and novel arrangements of elements and methods that are configured in unique and novel ways and which demonstrate previously unavailable but preferred and desirable capabilities.

The detailed description set forth below in connection with the drawings is intended merely as a description of the presently preferred embodiments of the invention, and is not intended to represent the only form in which the present invention may be constructed or utilized. The description sets forth the designs, functions, means, and methods of implementing the invention in connection with the illustrated embodiments. It is to be understood, however, that the same or equivalent functions and features may be accomplished by different embodiments that are also intended to be encompassed within the spirit and scope of the invention.

Derivation of an Extremely Platelet Rich Plasma (EPRP) as a Precursor to the Instant Invention

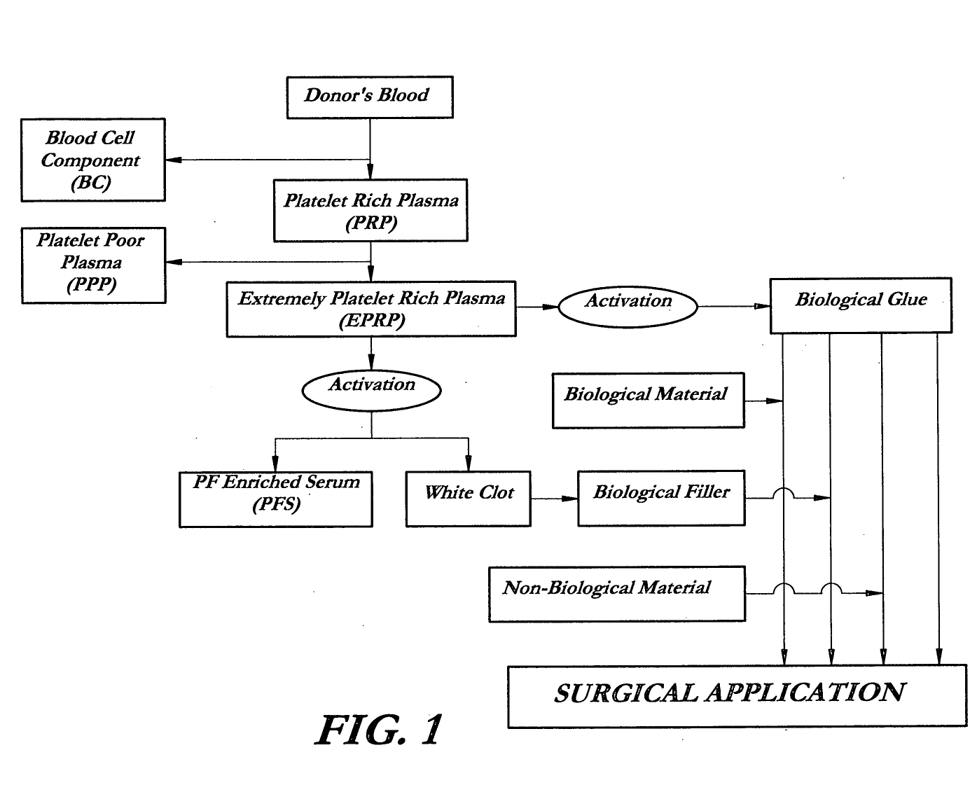

Initially, as seen in FIG. 1, blood may be obtained by venipuncture of a predetermined subject, may be obtained from blood previously banked by a predetermined subject (autologous), or may be obtained from banked blood from random or selected subjects (heterologous). According to techniques well known in the art, a unit of whole blood (500 cc) may be centrifuged to yield approximately 250 cc of Platelet Rich Plasma (PRP), with the remainder being a blood-cell component (BC) that is substantially composed of erythrocytes, leukocytes, and some residual plasma. The Blood Cell (BC) component is removed from further use in the method and may be utilized in a myriad of other applications, as would be well known to one skilled in the art. The Platelet Rich Plasma (PRP) may be centrifuged at high speed, as would be known to one skilled in the art, to yield a pellet of platelet aggregate in approximately 50 cc of residual plasma, along with a few platelets. The relatively platelet free plasma component is removed from further use in the method and may be utilized in a myriad of other applications, as would be well know to one skilled in the art.

The 50 cc quanta of plasma containing the pellet of platelet aggregate is allowed to settle for 1 hour at ambient temperature, in order to favor platelet disaggregation and to create the Extremely Platelet Rich Plasma (EPRP). The platelets are then placed in a mechanical rotor to gently resuspend the platelet pellet. Most platelets originally present in the unit of whole blood are present in the Extremely Platelet Rich Plasma (EPRP). In order to keep the platelets in their optimum functional state they must be constantly shaken at room temperature (20-24° C.), and may be conserved for as long as 5 days if they are maintained at a pH of 6 or higher. Platelets from multiple units of blood may be combined to form Extremely Platelet Rich Plasma (EPRP); however, there should be a minimum of 5.5×10¹⁰ platelets in at least 75% of the units so combined.

Activation of the Extremely Platelet Rich Plasma (EPRP)

As further seen in FIG. 1, the Extremely Platelet Rich Plasma (EPRP) is activated to create the Platelet Factor Enriched Serum (PFS), Biological Glue 100, Biological Implant 200, and Biological Filler 150 of the instant invention. The Extremely Platelet Rich Plasma (EPRP) previously collected as detailed above can be activated by the following protocol: To each 9 milliliters of Extremely Platelet Rich Plasma (EPRP) collected as detailed above, between 1 and 2 milliliters of calcium glucobionate are added, each milliliter of calcium glucobionate providing 4.5 mg of elemental calcium. This solution is warmed to approximately 37° C. and may immediately be used as a Biological Glue 100, that is, it may be used to fill small defects by itself or as a form of glue or paint to cause adhesion of cells derived from pre-existing cell cultures, or other materials, including biologic or non-biological materials 140, or Biological Filler 150, to a biological surface. By way of example, and not limitation, the glue may be used with other natural or artificial material 140 implanted at a biological site, to enhance adhesion. As described above, the Biological Glue may be used to secure, by way of example and not limitation, various prosthetic implants, such as bone prostheses, osteosynthesis pieces, and dental prostheses. The Biological Glue may be injected between tissue layers in order to adhere such layers and possesses a volume that may be sufficient to fill small defects, as seen in FIGS. 13-17.

If the Biological Glue 100 is not used as such in a short period of time, a thrombus spontaneously forms, illustrated as the separation of PF Enriched Serum (PFS) and White Clot in FIG. 1. Removal of the thrombus results in a Platelet Factor Enriched Serum (PFS) rich in platelet derived factors (PDFs) and other compounds in the liquid portion. The Platelet Factor Enriched Serum (PFS) may be used to enhance the growth of cell cultures 250, discussed below, and is utilized in one embodiment of the instant invention. The clot may be further processed, for additional applications, including Biological Filler. By way of example, the clot may be freeze-dried and then ground, to provide a biologically based source for Biological Filler 150 material.

Addition of Specialized Cultured Cells or Other Materials to the Extremely Platelet Rich Plasma (EPRP) Prior to Activation

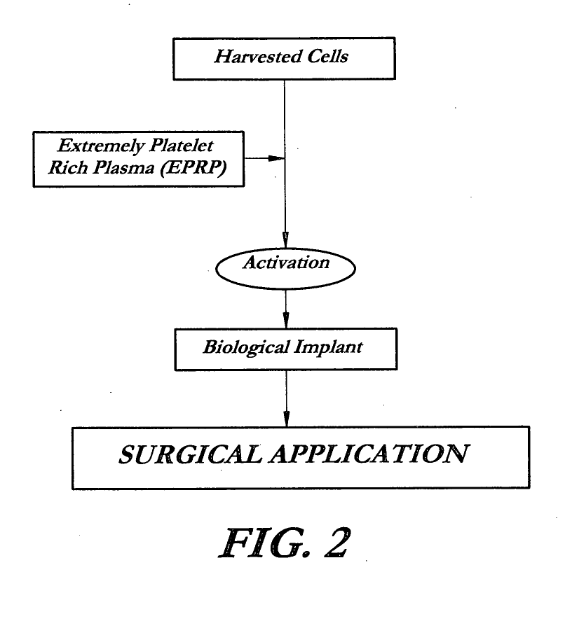

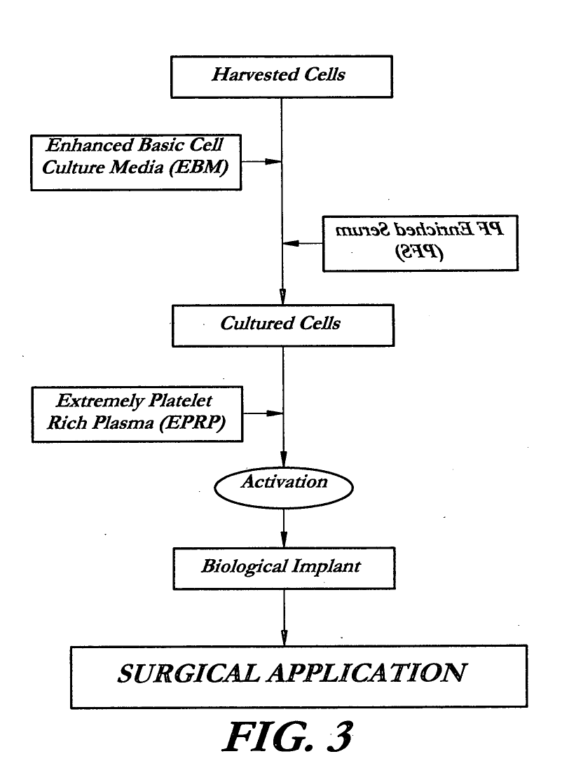

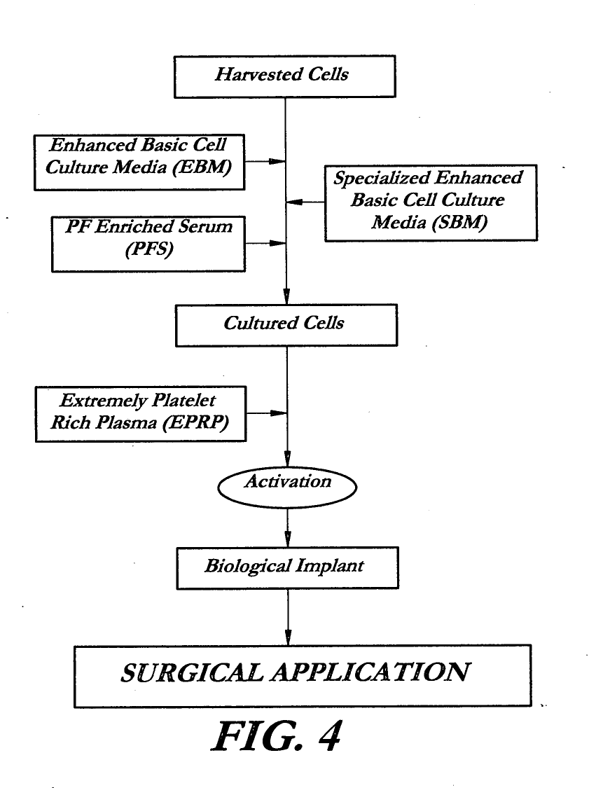

Alternatively, various materials, illustrated as Non-Biological Material, Biological Filler, and Biological Material, in FIG. 5, may be added to the Extremely Platelet Rich Plasma (EPRP) prior to activation. These may include various inert additives such as calcium carbonate, hydroxyapatite, or various biodegradable polymers. Freshly harvested cells may be mixed with Extremely Platelet Rich Plasma (EPRP) as seen in FIG. 2. In one embodiment, seen in FIGS. 3-4, the Extremely Platelet Rich Plasma (EPRP) is mixed with cultured cells 250 to produce a Biological Implant 200 that may be used to fill defects or potential spaces, and, having cells disposed throughout the matrix of the resulting implant, provides an ideal structural and nutritional environment for the growth and proliferation of such cells.

Enhanced Basic Cell Culture Media (EBM)

Cells being prepared for implantation, as seen in FIG. 3, as part of a Biological Implant 200 of the instant invention grow well in a culture media comprising commercial cell culture media supplemented with amino acids, antibiotics and fungicides, biological response modifiers, hormones, inorganic salts, metabolic intermediates, and vitamins. The group of amino acids may, by way of example only, include such amino acids as L-Glutamine, L-Histidine, L-Methionine, L-Phenylalanine, L-Tryptophan, L-Tyrosine, and L-Isoleucine. The group of antibiotics and fungicides may, by way of example only, include such antibiotics and fungicides as penicillin, streptomycin, and amphotericin B. The group of biological response modifiers, by which it is meant compounds that affect physiologic responses, may, by way of example only, include such biological response modifiers as sodium heparin and choleric toxin. The group of hormones may, by way of example only, include such hormones as glucagon, hydrocortisone, recombinant human insulin, and levothyroxine. The group of inorganic salts may, by way of example only, include such inorganic salts as sodium bicarbonate and sodium selenite. The group of metabolic intermediates may, by way of example only, include such metabolic intermediates as adenosine triphosphate, choline, cyticholine (histidine-5′-choline disphosphate), ethanolamine, linoleic acid, myo-inositol, oleic acid, para-amino benzoic acid, phosphoethanolamine, and sodium pyruvate. The group of vitamins may, by way of example only, include such vitamins as D-biotin, D-pantothenic acid, folic acid, niacinamide, pyridoxine, riboflavin, thiamine, and vitamin B12.

In one embodiment, a useful formulation is a standard commercially available cell culture media, such as Dulbecco's minimal essential media (DMEM), forming a base solution, and the following supplements are added in the following approximate ranges of concentrations, to produce an Enhanced Basic Cell Culture Media (EBM): adenosine triphosphate (1-10 mg/l), sodium bicarbonate (1.2-5 g/l), cyticholine (histidine-5′-choline disphosphate (0-100 mg/l), ethanolamine (10-50 mg/l), phosphoethanolamine (5-25 mcg/l), glucagon (1-5 mg/l), L-glutamine (2-10 mM), sodium heparin (10,000-50,000 IU/l), hydrocortisone (10-100 mg/l), recombinant human insulin (100-1000 IU/l), Levothyroxine (50-200 mcg/l), linoleic acid (10-50 mcg/l), oleic acid (10-50 mcg/l), sodium pyruvate (50-150 mg/l), sodium selenite (10-50 mg/l), choleric toxin (0.1-1.0 mg/l), penicillin (100,000 IU/l), streptomycin (100 mcg/l), amphotericin B (2.5 mcg/l), choline (1-30 mg/l), folic acid (1-10 mg/l), myo-inositol (1-40 mg/l), niacinamide (1-10 mg/l), p-amino benzoic acid (1-10 mg/l), D-pantothenic acid (1-15 mg/l), pyridoxine (1-10 mg/l), riboflavin 2-20 mg/l), thiamin (1-5 mg/l), vitamin B12 (1-10 mcg/l), L-histidine (1-20 mg/l), L-isoleucine (4-50 mg/l), L-methionine (1-25 mg/l), L-phenylalanine 2-20 mg/l), L-tryptophan, (1-5 mg/l), and L-tyrosine (2-10 mg/l). For human cell culture applications, human albumin (1,000 mg/l), and human transferin (50 mg/l) may be added.

Platelet Factor Enriched Serum (PFS) derived from prior activation of quanta of Extremely Platelet Rich Plasma (EPRP) by the method discussed above may be added to the Enhanced Basic Cell Culture Media (EBM) to provide platelet derived factors to the media. Platelet Factor Enriched Serum (PFS) is added in some embodiments to a final concentration in the cell culture media of approximately between 1-30%, with an approximate range of 5-10% being optimal in some embodiments. Cultured cells may also be dispersed in additional quanta of donor derived plasma prior to activation to form a Biological Implant.

Specialized Enhanced Basic Cell Culture Media (SBM)

Certain specialized cell lines have been found to benefit from additional supplements to the Enhanced Basic Cell Culture Media (EBM) described above, and as seen in FIG. 4. Such additions to the Enhanced Basic Cell Culture Media (EBM) create a Specialized Enhanced Basic Cell Culture Media (SBM) particularly adapted to various cells. The following additives have been found beneficial in the concentrations specified for these cell types:

For adipocytes; D-biotin (1-10 mg/l), and dexamethasone (1-10 mg/l).

For melanocytes; basic fibroblast growth factor (bFGF)(10-100 mcg/l), and theophylline (1-100 mg/l).

For chondrocytes and osteoblasts; L-ascorbic acid (20-100 mg/l), human recombinant calcitonin (100-10,000 IU/l), calcitrol (0.1-10 mcg/l), dexamethasone (1-10 mg/l), and inorganic salts such as monobasic anhydrous potassium phosphate (100-500 mg/l) and dibasic anhydrous potassium phosphate (1,000-2,500 mg/l) or equivalent salts.

For keratinocytes; recombinant human leukemia inhibition factor (1,000 IU/ml) and forskolin (0.1 mg/l).

In addition, cultures of adipocytes, melanocytes, and chondrocytes have been found to benefit from the addition of dexamethasone to the culture media.

Culture and Maintenance of the Cell Lines

Enhanced Basic Cell Culture Media (EBM) or Specialized Enhanced Basic Cell Culture Media (SBM), depending on the cell type to be cultured, was prepared according to the specifications detailed above. The pH was adjusted to 7.40 and the osmolarity for human cells of the culture was 290 mOsm/kg, assumed to be ideal for the maintenance in vitro of human cell cultures. As is known in the art, this osmolarity varies with species, as for example an optimal osmolarity for murine cell culture is 310 mOsm/kg.

The cells were kept in culture flasks of 25 cm² or 75 cm² in appropriate culture media with the Platelet Factor Enriched Serum (PFS) containing products of platelet degranulation in an incubator at approximately 37° C. with an approximately 5% CO₂ atmosphere. These cell cultures 250 were found viable and suitable for implantation according to the instant invention after more than 90 days of incubation.

When the cells contained in a culture flask were adhered to the flask surface, forming a monolayer (exponential growth stage), the cells were obtained by the following process:

First, the culture media was decanted from the flask. Second, the remaining cells were washed with a phosphate buffered saline solution (pH 7.3 at 4° C.), and the wash solution was decanted. Third, a solution of EDTA-PBS in a concentration of 1-10 mM was added and vigorously shaken in order to release the cells adhered to the bottom of the flask. Fourth, phosphate buffered saline was added and this suspension was then centrifuged at 200 times the force of gravity (g) for 5 minutes at 37° C. Lastly, the supernatant was decanted and the remaining pellet of cells was resuspended in PBS.

Cellular viability studies were conducted using the exclusion method with trypan blue at 0.1% in a corpuscle counting device. If the number of viable cells was above 95%, the cells were again resuspended in the required volume to obtain a cell concentration of approximately 5×10⁵ viable cells in 0.1 ml of PBS. All of the above steps were performed in sterile conditions using sterile flow laminar hoods.

In order to detect possible contamination by mycoplasma, the cultures were periodically subjected to a fluorescence mycoplasma detection test according to the following protocol. Cells maintained in the culture were fixed for 30 minutes in the dark and at ambient temperature in methanol and acetic acid (3:1 ratio) and submerged in a buffered Hoechst 3258 solution (bisbenzimidazol) prepared from a sterile stock solution consisting of 100 ml of PBS and 15 mg of Hoechst 3258, with later dilution to 1:500 in PBS. The cells were observed in a fluorescence microscope and, in the event of contamination by mycoplasma, small fluorescent bodies were seen in the extranuclear and intercellular space.

Compatibility of Enhanced Basic Cell Culture Media (EBM) and Specialized Enhanced Basic Cell Culture Media (SBM) with Maintenance of Cell Lines at Low Temperatures

Cell cultures 250 prepared and maintained according to the above protocol were found to be highly suitable for long term storage at low temperatures. After determination of a concentration of viable cells greater than 95% according to the trypan blue protocol detailed above, cell cultures 250 destined for long term storage are resuspended in a solution of the appropriate culture medium and 10% dimethylsulfoxide (DMSO). The concentration was adjusted to a cell concentration of approximately 2×10⁶ cells/ml. The suspension was introduced into freezing vials and submerged in liquid nitrogen. They were subsequently stored in liquid nitrogen containers. The cells were thawed by heating the vials at approximately 37° C. with a later centrifugation to eliminate the residual DMSO. The cellular pellet was then resuspended in whole culture media and seeded into a culture flask, and in this manner introduced into an incubation oven at approximately 37° C.

Compatibility of Enhanced Basic Cell Culture Media (EBM) and Specialized Enhanced Basic Cell Culture Media (SBM) with Maintenance of Stem Cell Lines

After selecting the stem cells to be cultured, the maintenance of these stem cells in the basic culture medium may be conditioned by STO cells, such as STO SNL 76/7 or VERO for 48 hours, and up to 40% of this conditioned medium added to the Enhanced Basic Cell Culture Media (EBM) or Specialized Enhanced Basic Cell Culture Media (SBM) used for cell cultures. STO SNL 76/7 cells or VERO cells in the form of mitotically inactivated monolayers have been used in standard procedures for this type of culture. These monolayers can secrete embryotrophic substances such as transforming growth factor a (TGF-a), transforming growth factor b (TGF-b), platelet derived growth factor a (PDGF-a), and insulin type growth factors I and II (IGF-1 and IGF-2), and thus support both embryo development and stem cell maintenance. Human recombinant leukemia inhibiting factor (LIF) may be used to maintain the pluripotent phenotype of stem cells. In order to obtain levels which allow formation of stem cell niches which remain undifferentiated, this factor, LIF, can on its own maintain and serve to isolate stem cells.

By way of example, stem cells derived from pilosebaceous units show embryonic cell characteristics, such as a positive alkaline phosphatase activity similar to blastocytes. Such activity serves as a marker that allows detection and identification of the quality of stem cell culture.

Cultured stem cells 250 may be isolated by an enzymatic process with trypsin and/or DNAase depending on the cell type. The cell lines are placed in a culture flask with 25 cm² of surface area and the subcultures observed for the presence of embryoid bodies and the migration of monolayer cells into embryoid bodies.

Preparation of Cells for Implantation

Cells to be used for implantation in the Biological Implant 200 of the instant invention may be prepared in the following manner: The cell line is trypsinized and the trypsin is inactivated with an inactivator and the cells are centrifuged at 200 g. The supernatant is removed and the cell pellet is resuspended in 1 ml of enhanced culture medium. An aliquot containing approximately 10⁷ cells is prepared in 1 ml Extremely Platelet Rich Plasma (EPRP) and implant recipient derived plasma to make a final volume of 10 ml. The entire procedure is performed at 37° C.

In the case of osteoblasts, variable layers may be produced in culture in which calcification can be induced in varying degree. The extracellular mesh produced by the osteoblasts in culture made in vitro presents a varying degree of mineralization, so that it allows introduction of autografts or allografts into osteoarticular defects in order to cover or fill in bone defects, as seen in FIG. 8.

### INDUSTRIAL APPLICABILITY

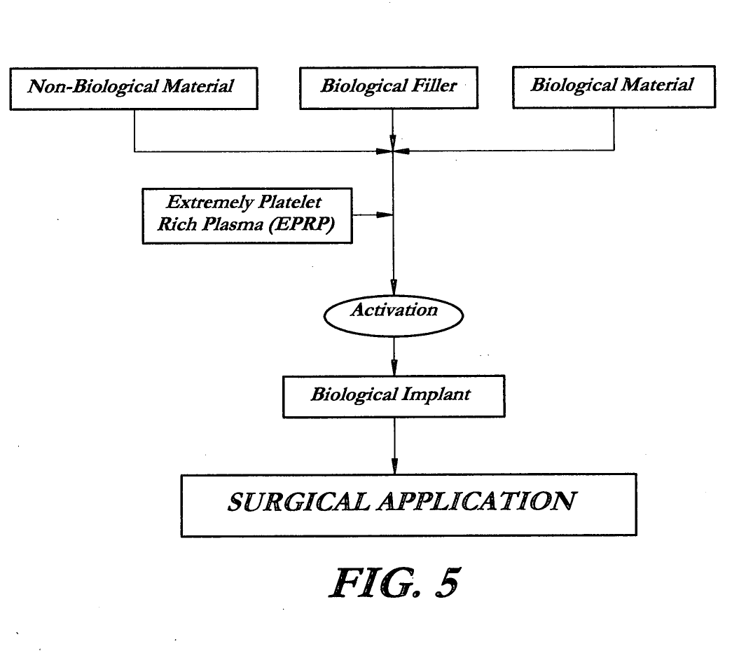

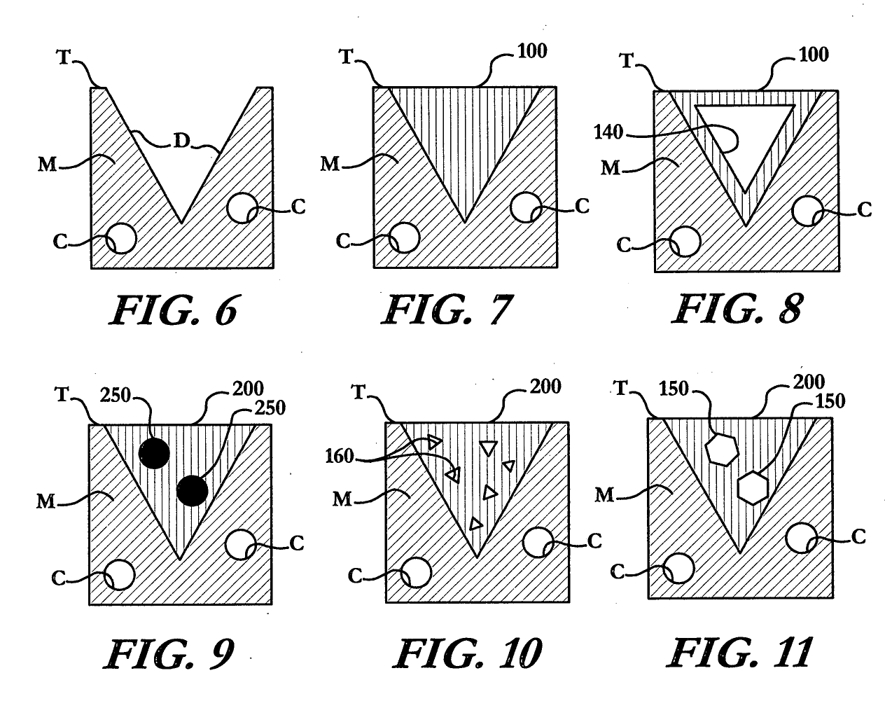

Platelet factors released into the Biological Glue 100 and Biological Implant 200 achieve an increase in cell proliferation and encouraged cell growth. Simultaneously, the Biological Glue 100 and Biological Implant 200 provide a physical environment that adheres itself, and any cells present, to the exposed surfaces of a wound or defect, as illustrated in FIGS. 6-11, and thereby prevents degradation of the site.

Bioimplants can be used in the following manners. The newly prepared Biological Glue 100 can serve as a material for direct adhesion of other organic or inorganic material on the site of the wound or implant, as seen in FIG. 8. Alternatively, a Biological Implant 200 can serve as a carrier for cells, such as for example, as seen in FIG. 9 and FIG. 15, to carry retinal pigment epithelium cells obtained from culture 250 or those previously conserved in liquid nitrogen, and to adhere those cells in the area of a retinal detachment. If a defect is small, the glue itself will serve to fill small defects, as seen in FIG. 7.

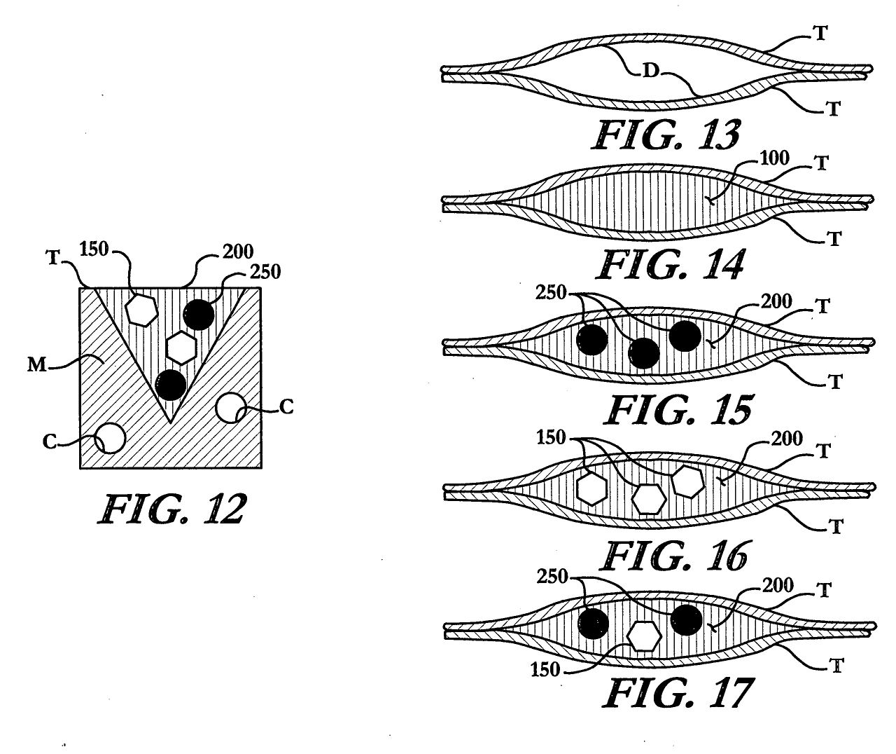

Biological Glue 100 may also be used to prepare a site for implantation. The wound or defect can be soaked in the Biological Glue 100 and an implant later compressed at the site of the wound. Within 48 hours, cell proliferation is observed and, in the case of repaired bone defects implanted with osteoblasts, after 3 weeks calcification nodules can be observed. The speed with which a thrombus is formed in the activation of the Biological Glue 100 results in cells being quickly adhered to wound or defect surfaces. For example, these cells may include fibroblasts implanted in the dermal or hypodermal region or pigment epithelium cells in the retina. The case of implanting pigment epithelium cells in the retina deserves special attention as the final volume of Biological Glue 100 when the thrombus forms is almost negligible (1 mm³ on the average). As these cells have considerable regenerative power due to their pluripotent nature, this method may be applied to retinal regeneration in such diseases as retinitis pigmentosa or retinal detachment. Analogous to the open defects illustrated in FIGS. 6-12, the use of Biological Glue 100 and Biological Implants 150 may be adapted to closed or interstitial defects, such as those illustrated in FIGS. 13-17.

Thrombus produced that is later frozen, desiccated, or freeze dried has a usable storage life of more than three years. Desiccated products may be irrigated with the Biological Glue 100, and such suspensions may be applied to the wound or defect to be treated. The method of the instant invention is well suited to the use of autologous cells, which minimizes concerns of tissue reaction and rejection. Accordingly, the Enhanced and Specialized Enhanced Basic Cell Culture Media (EBM and SBM), Biological Glue 100, Biological Implant 200 and Biological Filler 150 material can be used in the treatment of traumatic or surgical wounds, in bone implants or osteoarticular reconstructions, in maintenance of various grafts, and in long term in vitro maintenance of Biological Implants 200. The instant invention allows a single enhanced cell growth media to be prepared in advance of use, that may, depending on the intended use, be supplanted with specific supplements designed for the application intended. This media may be combined with cells, harvested or cultured, 250.

### EXEMPLARY IMPLEMENTATION OF THE INSTANT INVENTION

1. Creation of a Dermocutaneous Substitute with a Capacity to Develop Pilous Follicles

Cell lines are obtained from a skin punch of variable diameter or from the follicle unit or the pilous sebaceous unit. The skin specimens may be cut into portions, obtaining keratinocytes and melanocytes from the epidermis, fibroblasts from the dermis, and adipocytes from the hypodermis. These are cultured in a specialized cell culture media created by adding dexamethasone to Enhanced Basic Cell Culture Media (EBM) as previously described. The adipocytes are obtained by enzymatic processing with dipase/collagenase. Approximately 10⁷ cells are resuspended in 5 ml of fresh culture media, and then cultured as previously described. Fibroblasts are extracted in the same manner. Finally, the keratinocytes may be cultivated in a media enhanced with recombinant human leukemia inhibition factor, (1,000 IU/ml), and forskolin (0.1 mg/l) to maintain the specificity of the cutaneous stem cells.

2. Creation of a Biological Implant Containing Osteoblasts

Cells are obtained from trabecular bone from a bone crest or by bone puncture. The sample is washed well to remove remains of bone marrow or perisoteum. The cells are cultured in a Specialized Enhanced Basic Cell Culture Media (SBM) formed by adding L-ascorbic acid, calcitrol, recombinant human calcitonin, and inorganic salts to Enhanced Basic Cell Culture Media (EBM).

Osteoarticular defects of varying etiologies are cleaned and Biological Glue 100 may be applied to the defect, filling small defects as seen in FIG. 7. Alternatively, the defect may be shaped into a rough shape in accordance with additional material to be implanted. Biological Glue 100 may be used to adhere organic or inorganic materials to the defect, as seen in FIG. 8. Additionally, osteoblasts or other material may be incorporated into a Biological Implant 200 according the protocol above, that is, by mixing cultured cells 250 or other material into the Extremely Platelet Rich Plasma (EPRP) prior to activation, as seen in FIG. 9 and FIG. 10. The added material may include Biological Filler 150 derived from prior activation of Biological Glue 100, as seen in FIG. 11. Upon activation, the mass will begin to coagulate, and the Biological Implant 200 may be placed in the defect.

3. Creation of a Biological Implant Containing Chondrocytes

Specimens may be recovered from articular cartilage and then chondrocytes liberated from the specimens by processing with dipase/collagenase. The chondrocytes may be cultured in a Specialized Enhanced Basic Cell Culture Media (SBM) formed by adding L-ascorbic acid, calcitrol, and inorganic salts to Enhanced Basic Cell Culture Media (EBM). The chondrocytes are resuspended in culture media in a concentration of approximately 10⁷ cells/ml. The damaged area of cartilage in the intended recipient may be reached by arthroscopic surgery. A Biological Implant 200 made of cultured cells added to the Extremely Platelet Rich Plasma (EPRP) will quickly begin to coagulate upon activation and may be immediately implanted in the cartilaginous defect, where coagulation and adhesion will rapidly conclude.

4. Biological Implant for Repairing Retinal Detachment and/or Regeneration in the Case of Retinitis Pigmentosa

Cells recovered from retinal pigment epithelium may be cultured in the Enhanced Basic Cell Culture Media (EBM). Cultured cells 250 may be resuspended as detailed above and then added to the Extremely Platelet Rich Plasma (EPRP) to provide a Biological Implant 200 that is approximately 1 ml in total volume. This implant may then be injected into the area of retinal detachment or degeneration, as illustrated in FIGS. 13-15. Other types of closed or interstitial defects may be repaired according to the instant invention as seen in FIGS. 13-17.

5. Creation of Other Biological Implants

Other types of Biological Implants 200 that may be created include, but are not limited to, implants bearing melanocytes. For experimental purposes in animals, a Biological Implant 200 bearing tumor cells may be prepared and implanted according to the procedures of the instant invention.

What is claimed then, is a method for biological tissue repair in a recipient, comprising the steps of extracting an Extremely Platelet Rich Plasma (EPRP) from whole blood; activating coagulation of the Extremely Platelet Rich Plasma (EPRP) by the addition of Calcium wherein the activation is carried out in an environment free of exogenous thrombin; and placing at least a portion of the activated Extremely Platelet Rich Plasma (EPRP) at a biological site of the intended recipient.

In an embodiment of the instant invention, the activated Extremely Platelet Rich Plasma (EPRP) is allowed to form a thrombus and then the thrombus is removed from the activated Extremely Platelet Rich Plasma (EPRP) to leave a Platelet Factor Enriched Serum (PFS). The thrombus may be preserved and reserved for later use.

In an additional embodiment, biological material may be added to the Extremely Platelet Rich Plasma (EPRP) prior to activation to form a Biological Implant that is implanted in the intended recipient subsequent to activation to at least partially occupy a space, or, or in addition to which, autologous plasma derived from the intended recipient maybe added to the Extremely Platelet Rich Plasma (EPRP) prior to activation.

The autologous plasma may be added in a concentration of between approximately 1 and approximately 30 volume percent, or in an alternate embodiment, the autologous plasma may be added in a concentration of between approximately 5 and approximately 10 volume percent.

In addition to, or as an alternate to, adding biological material, non-biological material may be added to the Extremely Platelet Rich Plasma (EPRP) prior to activation to form a Biological Implant that is implanted in the intended recipient subsequent to activation to at least partially occupy a space. In an embodiment, such non-biological material added to the Extremely Platelet Rich Plasma (EPRP) may be a matrix former selected from the group consisting of calcium carbonate, hydroxyapatite, and biodegradable polymer. In another embodiment, the biological material may be a portion of at least one processed thrombus. In yet another embodiment, the biological material may be a plurality of cells selected from the group of cells consisting of autologous cells harvested from the intended recipient, and heterologous cells selected for minimal immune reaction to the intended recipient. In embodiments adding a plurality of cells, the plurality of cells may be further selected from the group of cells further comprising a plurality of tumor cells and stem cells. Such cells may be cultured in a cell culture media including minimal essential media and the Platelet Factor Enriched Serum (PFS) derived from the Biological Glue, and the minimal essential media may further be Dulbecco's minimal essential media (DMEM).

In another embodiment, the cell culture media further is an Enhanced Basic Cell Culture Media (EBM) comprising a plurality of amino acids; antibiotics and fungicides; biological response modifiers; hormones; inorganic salts; metabolic intermediates; and vitamins.

In yet another embodiment, the cell culture media may further comprise human albumin present in a concentration of substantially 1,000 mg/l and human transferin present in a concentration of substantially 50 mg/l. In another embodiment of EBM, the plurality of amino acids may be selected from the group consisting of: L-Glutamine; L-Histidine; L-Methionine; L-Phenylalanine; L-Tryptophan; L-Tyrosine; and L-Isoleucine.

In an alternate embodiment of EBM, the amino acids may be selected from the group consisting of: L-Glutamine present in a concentration from approximately 2 to approximately 10 mM/l; L-Histidine present in a concentration from approximately 1 to approximately 20 mg/l; L-Methionine present in a concentration from approximately 1 to approximately 25 mg/l; L-Phenylalanine present in a concentration from approximately 2 to approximately 20 mg/l; L-Tryptophan present in a concentration from approximately 1 to approximately 5 mg/l; L-Tyrosine present in a concentration from approximately 2 to approximately 10 mg/l; and L-Isoleucine present in a concentration from approximately 4 to approximately 50 mg/l.

In a preferred embodiment of EBM, the amino acids may be selected from the group consisting of: L-Glutamine present in a concentration of approximately 2 mM/l; L-Histidine present in a concentration of approximately 2 mg/l; L-Methionine present in a concentration of approximately 1 mg/l; L-Phenylalanine present in a concentration of approximately 2 mg/l; L-Tryptophan present in a concentration of 1 approximately mg/l; L-Tyrosine present in a concentration of approximately 2 mg/l; and L-Isoleucine present in a concentration of approximately 4 mg/l.

In another embodiment of EBM, the antibiotics and fungicides may be selected from the group consisting of: penicillin; streptomycin; and amphotericin B. In a preferred embodiment of EBM, the antibiotics and fungicides may be selected from the group consisting of: penicillin present in a concentration of approximately 100,000 IU/l; streptomycin present in a concentration of approximately 100 mcg/l; and amphotericin B present in a concentration of approximately 2.5 mcg/l.

In an alternate embodiment of EBM, the biological response modifiers may be selected from the group consisting of sodium heparin and choleric toxin. In a further embodiment of EBM, the biological response modifiers may be selected from the group consisting of sodium heparin present in a concentration from approximately 10,000 to approximately 50,000 IU/l and choleric toxin present in a concentration from approximately 0.1 to approximately 1.0 mg/l. In a preferred embodiment of EBM, the biological response modifiers may be selected from the group consisting of sodium heparin present in a concentration of approximately 10,000 IU/l and choleric toxin present in a concentration of approximately 0.1 mg/l.

In an embodiment of EBM, the hormones may selected from the group consisting of: glucagon; hydrocortisone; recombinant human insulin; and levothyroxine. In another embodiment of EBM, the hormones may be selected from the group consisting of: glucagon present in a concentration from approximately 1 to approximately 5 mg/l; hydrocortisone present in a concentration from approximately 10 to approximately 100 mg/l; recombinant human insulin present in a concentration from approximately 100 to approximately 1000 IU/l; and levothyroxine present in a concentration from approximately 50 to approximately 200 mcg/l.

In a preferred embodiment of EBM, the hormones may be selected from the group consisting of: glucagon present in a concentration of approximately 1 mg/l; hydrocortisone present in a concentration of approximately 10 mg/l; recombinant human insulin present in a concentration of approximately 100 IU/l; and levothyroxine present in a concentration of approximately 50 mcg/l.

In another embodiment of EBM, the inorganic salts may be selected from the group consisting of sodium bicarbonate and sodium selenite. In yet another embodiment of EBM, the inorganic salts may selected from the group consisting of sodium bicarbonate present in a concentration from approximately 1.2 to approximately 5 g/l and sodium selenite present in a concentration from approximately 10 to approximately 50 mg/l. In a preferred embodiment of EBM, the inorganic salts may be selected from the group consisting of sodium bicarbonate present in a concentration of approximately 1.2 g/l and sodium selenite present in a concentration of approximately 10 mg/l.

In an embodiment of EBM, the metabolic intermediates may be selected from the group consisting of: adenosine triphosphate; choline; cyticholine (histidine-5′-choline disphosphate); ethanolamine; linoleic acid; myo-inositol; oleic acid; para-amino benzoic acid; phosphoethanolamine; and sodium pyruvate. In a further embodiment of EBM, the metabolic intermediates may be selected from the group consisting of: adenosine triphosphate present in a concentration from approximately 1 to approximately 10 mg/l; choline present in a concentration from approximately 1 to approximately 30 mg/l; cyticholine (histidine-5′-choline disphosphate) present in a concentration from approximately 10 to approximately 100 mg/l); ethanolamine present in a concentration from approximately 10 to approximately 50 mg/l; linoleic acid present in a concentration from approximately 10 to approximately 50 mcg/l; myo-inositol present in a concentration from approximately 1 to approximately 40 mg/l; oleic acid present in a concentration from approximately 10 to approximately 50 mcg/l; para-amino benzoic acid present in a concentration from approximately 1 to approximately 10 mg/l; phosphoethanolamine present in a concentration from approximately 5 to approximately 25 mcg/l; and sodium pyruvate present in a concentration from approximately 50 m to approximately 150 mg/l.

In a preferred embodiment of EBM, the metabolic intermediates may be selected from the group consisting of: adenosine triphosphate present in a concentration of approximately 1 mg/l; choline present in a concentration of approximately 1 mg/l; cyticholine (histidine-5′-choline disphosphate) present in a concentration of approximately 10 mg/l; ethanolamine present in a concentration of approximately 10 mg/l; linoleic acid present in a concentration of approximately 10 mcg/l; myo-inositol present in a concentration of approximately 1 mg/l; oleic acid present in a concentration of approximately 10 mcg/l; para-amino benzoic acid present in a concentration of approximately 1 mg/l; phosphoethanolamine present in a concentration of approximately 5 mcg/l; and sodium pyruvate present in a concentration of approximately 50 mg/l.

In an embodiment of EBM, the vitamins may be selected from the group consisting of: D-biotin; D-pantothenic acid; folic acid; niacinamide; pyridoxine;
riboflavin; thiamine; and vitamin B12. In a further embodiment of EBM, the vitamins may be selected from the group consisting of: D-biotin present in a concentration from approximately 1 to approximately 10 mg/l; D-pantothenic acid present in a concentration from approximately 1 to approximately 15 mg/l; folic acid present in a concentration from approximately 1 to approximately 10 mg/l; niacinamide present in a concentration from approximately 1 to approximately 10 mg/l; pyridoxine present in a concentration from approximately 1 to approximately 10 mg/l; riboflavin present in a concentration from approximately 2 to approximately 20 mg/l; thiamine present in a concentration from approximately 1 to approximately 5 mg/l; and vitamin B12 present in a concentration from approximately 1 to approximately 10 mcg/l.

In a preferred embodiment of EBM, the vitamins may be selected from the group consisting of: D-biotin present in a concentration of approximately 1 mg/l; D-pantothenic acid present in a concentration of approximately 1 mg/l; folic acid present in a concentration of approximately 1 mg/l; niacinamide present in a concentration of approximately 1 mg/l; pyridoxine present in a concentration of approximately 1 mg/l; riboflavin present in a concentration of approximately 2 mg/l; thiamine present in a concentration of approximately 1 mg/l; and vitamin B12 present in a concentration of approximately 1 mcg/l.

In yet another embodiment, the Enhanced Basic Cell Culture Media (EBM) further comprises dexamethasone present in a concentration of approximately 1 to approximately 10 mg/l. In a further embodiment the EBM further comprises dexamethasone present in a concentration of approximately 1 mg/l.

In an embodiment of the instant invention, the Enhanced Basic Cell Culture Media (EBM) is a Specialized Enhanced Basic Cell Culture Media (SBM) specially adapted for the culture of melanocytes that further comprises: basic fibroblast growth factor (bFGF) present in a concentration of approximately 10 to approximately 100 mcg/l; and theophylline present in a concentration of approximately 1 to approximately 100 mg/l.

In yet another embodiment of the instant invention, the Enhanced Basic Cell Culture Media (EBM) is a Specialized Enhanced Basic Cell Culture Media (SBM) specially adapted for the culture of osteoblasts that further comprises: L-ascorbic acid present in a concentration of approximately 20 to approximately 100 mg/l; human recombinant calcitonin present in a concentration of approximately 100 to approximately 10,000 IU/l; calcitrol present in a concentration of approximately 0.1 to approximately 10 mcg/l; and at least one inorganic salt. In a further embodiment of SBM specially adapted for the culture of osteoblasts, the at least one inorganic salt may selected from the group consisting of monobasic anhydrous potassium phosphate and dibasic anhydrous potassium phosphate. Monobasic anhydrous potassium phosphate may further be in a concentration of approximately 100 to approximately 500 mg/l and dibasic anhydrous potassium phosphate may further be in a concentration of approximately 1,000 to approximately 2,500 mg/l.

In another embodiment of the instant invention, the Enhanced Basic Cell Culture Media (EBM) is a Specialized Enhanced Basic Cell Culture Media (SBM) specially adapted for the culture of chondrocytes that further comprises: L-ascorbic acid present in a concentration of approximately 20 mg/l; calcitrol (0.5 mcg/l); and at least one inorganic salt. In a further embodiment, the at least one inorganic salt may selected from the group consisting of monobasic anhydrous potassium phosphate and dibasic anhydrous potassium phosphate.

Monobasic anhydrous potassium phosphate may be further in a concentration of approximately 100 to approximately 500 mg/l and Dibasic anhydrous potassium phosphate may further be in a concentration of approximately 1,000 to approximately 2,500 mg/l.

In yet another embodiment of the instant invention, the Enhanced Basic Cell Culture Media (EBM) is a Specialized Enhanced Basic Cell Culture Media (SBM) specially adapted for the culture of stem cells that further comprises: human recombinant leukemia inhibiting factor present in a concentration of approximately 100 to approximately 10,000 IU/ml; thymidine present in a concentration of approximately 5 to approximately 10 mg/l; guanosine present in a concentration of approximately 10 to approximately 50 mg/l; uridine present in a concentration of approximately 10 to approximately 50 mg/l); 2-b-mercaptoethanolamine present in a concentration of approximately 10 to approximately 100 mcg/l; and forskolin present in a concentration of approximately 0.1 to approximately 10 mg/l.

In another embodiment of the instant invention, the Enhanced Basic Cell Culture Media (EBM) is a Specialized Enhanced Basic Cell Culture Media (SBM) specially adapted for the culture of keratinocytes and further comprising recombinant human leukemia inhibition factor present in a concentration of approximately 1,000 IU/l and forskolin present in a concentration of approximately 0.1 mg/l.

In an embodiment of the instant invention, cell culture media may further comprise: L-Glutamine present in a concentration from approximately 2 to approximately 10 mM/l; L-Histidine present in a concentration from approximately 1 to approximately 20 mg/l; L-Methionine present in a concentration from approximately 1 to approximately 25 mg/l; L-Phenylalanine present in a concentration from approximately 2 to approximately 20 mg/l; L-Tryptophan present in a concentration from approximately 1 to approximately 5 mg/l; L-Tyrosine present in a concentration from approximately 2 to approximately 10 mg/l; L-Isoleucine present in a concentration from approximately 4 to approximately 50 mg/l; penicillin present in a concentration of approximately 100,000 IU/l; streptomycin present in a concentration of approximately 100 mcg/l; amphotericin B present in a concentration of approximately 2.5 mcg/l; sodium heparin present in a concentration from approximately 10,000 to approximately 50,000 IU/l; choleric toxin present in a concentration from approximately 0.1-approximately 1.0 mg/l; glucagon present in a concentration from approximately 1 to approximately 5 mg/l; hydrocortisone present in a concentration from approximately 10 to approximately 100 mg/l; recombinant human insulin present in a concentration from approximately 100 to approximately 1000 IU/l; levothyroxine present in a concentration from approximately 50 to approximately 200 mcg/l; sodium bicarbonate present in a concentration from approximately 1.2 to approximately 5 g/l; sodium selenite present in a concentration from approximately 10 to approximately 50 mg/l; adenosine triphosphate present in a concentration from approximately 1 to approximately 10 mg/l; choline present in a concentration from approximately 1 to approximately 30 mg/l; cyticholine (histidine-5′-choline disphosphate) present in a concentration from approximately 10 to approximately 100 mg/l; ethanolamine present in a concentration from approximately 10 to approximately 50 mg/l; linoleic acid present in a concentration from approximately 10 to approximately 50 mcg/l; myo-inositol present in a concentration from approximately 1 to approximately 40 mg/l; oleic acid present in a concentration from approximately 10 to approximately 50 mcg/l; para-amino benzoic acid present in a concentration from approximately 1 to approximately 10 mg/l; phosphoethanolamine present in a concentration from approximately 5 to approximately 25 mcg/l; sodium pyruvate present in a concentration from approximately 50 to approximately 150 mg/l; D-biotin present in a concentration from approximately 1 to approximately 10 mg/l; D-pantothenic acid present in a concentration from approximately 1 to approximately 15 mg/l; folic acid present in a concentration from approximately 1 to approximately 10 mg/l; niacinamide present in a concentration from approximately 1 to approximately 10 mg/l; pyridoxine present in a concentration from approximately 1 to approximately 10 mg/l; riboflavin present in a concentration from approximately 2 to approximately 20 mg/l; thiamine present in a concentration from approximately 1 to approximately 5 mg/l; and vitamin B12 present in a concentration from approximately 1 to approximately 10 mcg/l.

In a preferred embodiment of the instant invention, the cell culture media may further comprise: L-Glutamine present in a concentration of approximately 2 mM/l; L-Histidine present in a concentration of approximately 2 mg/l; L-Methionine present in a concentration of approximately 1 mg/l; L-Phenylalanine present in a concentration of approximately 2 mg/l; L-Tryptophan present in a concentration of approximately 1 mg/l; L-Tyrosine present in a concentration of approximately 2 mg/l; L-Isoleucine present in a concentration of approximately 4 mg/l; penicillin present in a concentration of approximately 100,000 IU/l; streptomycin present in a concentration of approximately 100 mcg/l; amphotericin B present in a concentration of approximately 2.5 mcg/l; sodium heparin present in a concentration of approximately 10,000 IU/l; choleric toxin present in a concentration of approximately 0.1 mg/l; glucagon present in a concentration of approximately 1 mg/l; hydrocortisone present in a concentration of approximately 10 mg/l; recombinant human insulin present in a concentration of approximately 100 IU/l; levothyroxine present in a concentration of approximately 50 mcg/l; sodium bicarbonate present in a concentration of approximately 1.2 g/l; sodium selenite present in a concentration of approximately 10 mg/l; adenosine triphosphate present in a concentration of approximately 1 mg/l; choline present in a concentration of approximately 1 mg/l; cyticholine (histidine-5′-choline disphosphate) present in a concentration of approximately 10 mg/l; ethanolamine present in a concentration of approximately 10 mg/l; linoleic acid present in a concentration of approximately 10 mcg/l; myo-inositol present in a concentration of approximately 1 mg/l; oleic acid present in a concentration of approximately 10 mcg/l; para-amino benzoic acid present in a concentration of approximately 1 mg/l; phosphoethanolamine present in a concentration of approximately 5 mcg/l; sodium pyruvate present in a concentration of approximately 50 mg/l; D-biotin present in a concentration of approximately 1 mg/l; D-pantothenic acid present in a concentration of approximately 1 mg/l; folic acid present in a concentration of approximately 1 mg/l; niacinamide present in a concentration of approximately 1 mg/l; pyridoxine present in a concentration of approximately 1 mg/l; riboflavin present in a concentration of approximately 2 mg/l; thiamine present in a concentration of approximately 1 mg/l; and vitamin B12 present in a concentration of approximately 1 mcg/l.

In a further embodiment of the instant invention, a method for biological tissue repair in a recipient comprises the steps of: extracting an Extremely Platelet Rich Plasma (EPRP) from whole blood; activating coagulation of the Extremely Platelet Rich Plasma (EPRP) to form a Biological Glue wherein the activation is carried out in an environment free of exogenous thrombin; and placing at least a portion of the Biological Glue at a biological site of the intended recipient to adhere at least one material at the biological site of the intended implant recipient.

In yet another embodiment of the instant invention, a method for biological tissue repair in a recipient comprises the steps of: extracting an Extremely Platelet Rich Plasma (EPRP) from whole blood; adding a material to the Extremely Platelet Rich Plasma (EPRP) to substantially disperse the material throughout the Extremely Platelet Rich Plasma (EPRP); activating coagulation of the Extremely Platelet Rich Plasma (EPRP) to form a Biological Implant wherein the activation is carried out in an environment free of exogenous thrombin; and placing at least a portion of the Biological Implant at a biological site of the intended recipient to at least partially occupy a space.

Numerous alterations, modifications, and variations of the preferred embodiments disclosed herein will be apparent to those skilled in the art and they are all anticipated and contemplated to be within the spirit and scope of the instant invention. For example, although specific embodiments have been described in detail, those with skill in the art will understand that the preceding embodiments and variations can be modified to incorporate various types of substitute and or additional or alternative materials, and methods. Accordingly, even though only few variations of the present invention are described herein, it is to be understood that the practice of such additional modifications and variations and the equivalents thereof, are within the spirit and scope of the invention as defined in the following claims.

The corresponding structures, materials, acts, and equivalents of all means or step plus function elements in the claims below are intended to include any structure, material, or acts for performing the functions in combination with other claimed elements as specifically claimed.

We claim:

1. A method for biological tissue repair in a recipient, comprising the steps of:
extracting an Extremely Platelet Rich Plasma (EPRP) from whole blood;
activating coagulation of the Extremely Platelet Rich Plasma (EPRP) by the addition of Calcium; wherein the activation is carried out in an environment free of exogenous thrombin; and
placing at least a portion of the activated Extremely Platelet Rich Plasma (EPRP) at a biological site of the intended recipient.

2. The method of claim 1, further comprising the steps of allowing the activated Extremely Platelet Rich Plasma (EPRP) to form a thrombus and then removing the thrombus from the activated Extremely Platelet Rich Plasma (EPRP) to leave a Platelet Factor Enriched Serum (PFS).

3. The method of claim 2, further comprising the step of preserving the thrombus and reserving the removed thrombus for later use.

4. The method of claim 1, further comprising the step of adding biological material to the Extremely Platelet Rich Plasma (EPRP) prior to activation to form a Biological Implant that is implanted in the intended recipient subsequent to activation to at least partially occupy a space.

5. The method of claim 1, further comprising the step of adding autologous plasma derived from the intended recipient to the Extremely Platelet Rich Plasma (EPRP) prior to activation.

6. The method of claim 5, wherein the autologous plasma is added in a concentration of between approximately 1 and approximately 30 volume percent.

7. The method of claim 5, wherein the autologous plasma is added in a concentration of between approximately 5 and approximately 10 volume percent.

8. The method of claim 1, further comprising the step of adding non-biological material to the Extremely Platelet Rich Plasma (EPRP) prior to activation to form a Biological Implant that is implanted in the intended recipient subsequent to activation to at least partially occupy a space.

9. The method of claim 8, wherein the non-biological material added to the Extremely Platelet Rich Plasma (EPRP) is a matrix former selected from the group consisting of calcium carbonate, hydroxyapatite, and biodegradable polymer.

10. The method of claim 4, wherein the biological material is a portion of at least one processed thrombus.

11. The method of claim 4, wherein the biological material is a plurality of cells selected from the group of cells consisting of autologous cells harvested from the intended recipient, and heterologous cells selected for minimal immune reaction to the intended recipient.

12. The method of claim 11, wherein the plurality of cells are further selected from the group of cells further comprising a plurality of tumor cells and stem cells.

13. The method of claim 11, wherein the cells are cultured in a cell culture media including minimal essential media and the Platelet Factor Enriched Serum (PFS) derived from the Biological Glue.

14. The method of claim 13, wherein the minimal essential media is Dulbecco's minimal essential media (DMEM).

15. The method of claim 13, wherein the cell culture media further is an Enhanced Basic Cell Culture Media (EBM) comprising a plurality of:
amino acids;
antibiotics and fungicides;
biological response modifiers;
hormones;
inorganic salts;
metabolic intermediates; and
vitamins.

16. The method of claim 13, wherein the cell culture media further comprises human albumin present in a concentration of substantially 1,000 mg/l and human transferin present in a concentration of substantially 50 mg/l.

17. The method of claim 15, wherein the plurality of amino acids are selected from the group consisting of:
L-Glutamine;
L-Histidine;
L-Methionine;
L-Phenylalanine;
L-Tryptophan;
L-Tyrosine; and
L-Isoleucine.

18. The method of claim 15, wherein the amino acids are selected from the group consisting of:
L-Glutamine present in a concentration from approximately 2 to approximately 10 mM/l;
L-Histidine present in a concentration from approximately 1 to approximately 20 mg/l;
L-Methionine present in a concentration from approximately 1 to approximately 25 mg/l;
L-Phenylalanine present in a concentration from approximately 2 to approximately 20 mg/l;
L-Tryptophan present in a concentration from approximately 1 to approximately 5 mg/l;
L-Tyrosine present in a concentration from approximately 2 to approximately 10 mg/l; and
L-Isoleucine present in a concentration from approximately 4 to approximately 50 mg/l.

19. The method of claim 15, wherein the amino acids are selected from the group consisting of:
L-Glutamine present in a concentration of approximately 2 mM/l;
L-Histidine present in a concentration of approximately 2 mg/l;
L-Methionine present in a concentration of approximately 1 mg/l;
L-Phenylalanine present in a concentration of approximately 2 mg/l;
L-Tryptophan present in a concentration of 1 approximately mg/l;
L-Tyrosine present in a concentration of approximately 2 mg/l; and
L-Isoleucine present in a concentration of approximately 4 mg/l.

20. The method of claim 15, wherein the antibiotics and fungicides are selected from the group consisting of:
penicillin;
streptomycin; and
amphotericin B.

21. The method of claim 15, wherein the antibiotics and fungicides are selected from the group consisting of:
penicillin present in a concentration of approximately 100,000 IU/l;
streptomycin present in a concentration of approximately 100 mcg/l; and
amphotericin B present in a concentration of approximately 2.5 mcg/l.

22. The method of claim 15, wherein the biological response modifiers are selected from the group consisting of sodium heparin and choleric toxin.

23. The method of claim 15, wherein the biological response modifiers are selected from the group consisting of sodium heparin present in a concentration from approximately 10,000 to approximately 50,000 IU/l and choleric toxin present in a concentration from approximately 0.1 to approximately 1.0 mg/l.

24. The method of claim 15, wherein the biological response modifiers are selected from the group consisting of sodium heparin present in a concentration of approximately 10,000 IU/l and choleric toxin present in a concentration of approximately 0.1 mg/l.

25. The method of claim 15, wherein the hormones are selected from the group consisting of:
glucagon;
hydrocortisone;
recombinant human insulin; and
levothyroxine.

26. The method of claim 15, wherein the hormones are selected from the group consisting of:
glucagon present in a concentration from approximately 1 to approximately 5 mg/l;
hydrocortisone present in a concentration from approximately 10 to approximately 100 mg/l;
recombinant human insulin present in a concentration from approximately 100 to approximately 1000 IU/l; and
levothyroxine present in a concentration from approximately 50 to approximately 200 mcg/l.

27. The method of claim 15, wherein the hormones are selected from the group consisting of:
glucagon present in a concentration of approximately 1 mg/l;
hydrocortisone present in a concentration of approximately 10 mg/l;
recombinant human insulin present in a concentration of approximately 100 IU/l; and
levothyroxine present in a concentration of approximately 50 mcg/l.

28. The method of claim 15, wherein the inorganic salts are selected from the group consisting of sodium bicarbonate and sodium selenite.

29. The method of claim 15, wherein the inorganic salts are selected from the group consisting of sodium bicarbonate present in a concentration from approximately 1.2 to approximately 5 g/l and sodium selenite present in a concentration from approximately 10 to approximately 50 mg/l.

30. The method of claim 15, wherein the inorganic salts are selected from the group consisting of sodium bicarbonate present in a concentration of approximately 1.2 g/l and sodium selenite present in a concentration of approximately 10 mg/l.

31. The method of claim 15, wherein the metabolic intermediates are selected from the group consisting of:
adenosine triphosphate;
choline;
cyticholine (histidine-5′-choline disphosphate);
ethanolamine;
linoleic acid;
myo-inositol;
oleic acid;
para-amino benzoic acid;
phosphoethanolamine; and
sodium pyruvate.

32. The method of claim 15, wherein the metabolic intermediates are selected from the group consisting of:
adenosine triphosphate present in a concentration from approximately 1 to approximately 10 mg/l;
choline present in a concentration from approximately 1 to approximately 30 mg/l;
cyticholine (histidine-5′-choline disphosphate) present in a concentration from approximately 10 to approximately 100 mg/l);
ethanolamine present in a concentration from approximately 10 to approximately 50 mg/l;
linoleic acid present in a concentration from approximately 10 to approximately 50 mcg/l;
myo-inositol present in a concentration from approximately 1 to approximately 40 mg/l;
oleic acid present in a concentration from approximately 10 to approximately 50 mcg/l;
para-amino benzoic acid present in a concentration from approximately 1 to approximately 10 mg/l;
phosphoethanolamine present in a concentration from approximately 5 to approximately 25 mcg/l; and
sodium pyruvate present in a concentration from approximately 50 m to approximately 150 mg/l.

33. The method of claim 15, wherein the metabolic intermediates are selected from the group consisting of:
adenosine triphosphate present in a concentration of approximately 1 mg/l;
choline present in a concentration of approximately 1 mg/l;
cyticholine (histidine-5′-choline disphosphate) present in a concentration of approximately 10 mg/l;
ethanolamine present in a concentration of approximately 10 mg/l;
linoleic acid present in a concentration of approximately 10 mcg/l;
myo-inositol present in a concentration of approximately 1 mg/l;
oleic acid present in a concentration of approximately 10 mcg/l;
para-amino benzoic acid present in a concentration of approximately 1 mg/l;
phosphoethanolamine present in a concentration of approximately 5 mcg/l; and
sodium pyruvate present in a concentration of approximately 50 mg/l.

34. The method of claim 15, wherein the vitamins are selected from the group consisting of:
D-biotin;
D-pantothenic acid;
folic acid;
niacinamide;
pyridoxine;
riboflavin;
thiamine; and
vitamin B12.

35. The method of claim 15, wherein the vitamins are selected from the group consisting of:
D-biotin present in a concentration from approximately 1 to approximately 10 mg/l;
D-pantothenic acid present in a concentration from approximately 1 to approximately 15 mg/l;
folic acid present in a concentration from approximately 1 to approximately 10 mg/l;
niacinamide present in a concentration from approximately 1 to approximately 10 mg/l;
pyridoxine present in a concentration from approximately 1 to approximately 10 mg/l;
riboflavin present in a concentration from approximately 2 to approximately 20 mg/l;
thiamine present in a concentration from approximately 1 to approximately 5 mg/l; and
vitamin B12 present in a concentration from approximately 1 to approximately 10 mcg/l.

36. The method of claim 15, wherein the vitamins are selected from the group consisting of:
D-biotin present in a concentration of approximately 1 mg/l;
D-pantothenic acid present in a concentration of approximately 1 mg/l;
folic acid present in a concentration of approximately 1 mg/l;
niacinamide present in a concentration of approximately 1 mg/l;
pyridoxine present in a concentration of approximately 1 mg/l;
riboflavin present in a concentration of approximately 2 mg/l;
thiamine present in a concentration of approximately 1 mg/l; and
vitamin B12 present in a concentration of approximately 1 mcg/l.

37. The method of claim 15, wherein the Enhanced Basic Cell Culture Media (EBM) further comprises dexamethasone present in a concentration of approximately 1 to approximately 10 mg/l.

38. The method of claim 15, wherein the Enhanced Basic Cell Culture Media (EBM) further comprises dexamethasone present in a concentration of approximately 1 mg/l.

39. The method of claim 15, wherein the Enhanced Basic Cell Culture Media (EBM) is a Specialized Enhanced Basic Cell Culture Media (SBM) specially adapted for the culture of melanocytes and further comprising:
basic fibroblast growth factor (bFGF) present in a concentration of approximately 10 to approximately 100 mcg/l; and
theophylline present in a concentration of approximately 1 to approximately 100 mg/l.

40. The method of claim 15, wherein the Enhanced Basic Cell Culture Media (EBM) is a Specialized Enhanced Basic Cell Culture Media (SBM) specially adapted for the culture of osteoblasts and further comprising:
L-ascorbic acid present in a concentration of approximately 20 to approximately 100 mg/l;
human recombinant calcitonin present in a concentration of approximately 100 to approximately 10,000 IU/l;
calcitrol present in a concentration of approximately 0.1 to approximately 10 mcg/l; and
at least one inorganic salt.

41. The method of claim 40, wherein the at least one inorganic salt is selected from the group consisting of monobasic anhydrous potassium phosphate and dibasic anhydrous potassium phosphate.

42. The method of claim 41, wherein the at least one inorganic salt is monobasic anhydrous potassium phosphate in a concentration of approximately 100 to approximately 500 mg/l.

43. The method of claim 41, wherein the at least one inorganic salt is dibasic anhydrous potassium phosphate in a concentration of approximately 1,000 to approximately 2,500 mg/l.

44. The method of claim 15, wherein the Enhanced Basic Cell Culture Media (EBM) is a Specialized Enhanced Basic Cell Culture Media (SBM) specially adapted for the culture of chondrocytes and further comprising:
L-ascorbic acid present in a concentration of approximately 20 mg/l;
calcitrol (0.5 mcg/l); and
at least one inorganic salts.

45. The method of claim 44, wherein the at least one inorganic salt is selected from the group consisting of monobasic anhydrous potassium phosphate and dibasic anhydrous potassium phosphate.

46. The method of claim 45, wherein the at least one inorganic salt is monobasic anhydrous potassium phosphate in a concentration of approximately 100 to approximately 500 mg/l.

47. The method of claim 45, wherein the at least one inorganic salt is dibasic anhydrous potassium phosphate in a concentration of approximately 1,000 to approximately 2,500 mg/l.

48. The method of claim 15, wherein the Enhanced Basic Cell Culture Media (EBM) is a Specialized Enhanced Basic Cell Culture Media (SBM) specially adapted for the culture of stem cells and further comprising:
human recombinant leukemia inhibiting factor present in a concentration of approximately 100 to approximately 10,000 IU/ml;
thymidine present in a concentration of approximately 5 to approximately 10 mg/l;
guanosine present in a concentration of approximately 10 to approximately 50 mg/l;
uridine present in a concentration of approximately 10 to approximately 50 mg/l); 2-b-mercaptoethanolamine present in a concentration of approximately 10 to approximately 100 mcg/l; and
forskolin present in a concentration of approximately 0.1 to approximately 10 mg/l.

49. The method of claim 15, wherein the Enhanced Basic Cell Culture Media (EBM) is a Specialized Enhanced Basic Cell Culture Media (SBM) specially adapted for the culture of keratinocytes and further comprising recombinant human leukemia inhibition factor present in a concentration of approximately 1,000 IU/l and forskolin present in a concentration of approximately 0.1 mg/l.

50. The method of claim 13, wherein the cell culture media further comprises:
L-Glutamine present in a concentration from approximately 2 to approximately 10 mM/l;
L-Histidine present in a concentration from approximately 1 to approximately 20 mg/l;
L-Methionine present in a concentration from approximately 1 to approximately 25 mg/l;
L-Phenylalanine present in a concentration from approximately 2 to approximately 20 mg/l;
L-Tryptophan present in a concentration from approximately 1 to approximately 5 mg/l;
L-Tyrosine present in a concentration from approximately 2 to approximately 10 mg/l;
L-Isoleucine present in a concentration from approximately 4 to approximately 50 mg/l;
penicillin present in a concentration of approximately 100,000 IU/l;
streptomycin present in a concentration of approximately 100 mcg/l;
amphotericin B present in a concentration of approximately 2.5 mcg/l;
sodium heparin present in a concentration from approximately 10,000 to approximately 50,000 IU/l;
choleric toxin present in a concentration from approximately 0.1-approximately 1.0 mg/l;
glucagon present in a concentration from approximately 1 to approximately 5 mg/l;
hydrocortisone present in a concentration from approximately 10 to approximately 100 mg/l;
recombinant human insulin present in a concentration from approximately 100 to approximately 1000 IU/l;
levothyroxine present in a concentration from approximately 50 to approximately 200 mcg/l;
sodium bicarbonate present in a concentration from approximately 1.2 to approximately 5 g/l;
sodium selenite present in a concentration from approximately 10 to approximately 50 mg/l;
adenosine triphosphate present in a concentration from approximately 1 to approximately 10 mg/l;
choline present in a concentration from approximately 1 to approximately 30 mg/l;
cyticholine (histidine-5′-choline disphosphate) present in a concentration from approximately 10 to approximately 100 mg/l;
ethanolamine present in a concentration from approximately 10 to approximately 50 mg/l;
linoleic acid present in a concentration from approximately 10 to approximately 50 mcg/l;
myo-inositol present in a concentration from approximately 1 to approximately 40 mg/l;
oleic acid present in a concentration from approximately 10 to approximately 50 mcg/l;
para-amino benzoic acid present in a concentration from approximately 1 to approximately 10 mg/l;
phosphoethanolamine present in a concentration from approximately 5 to approximately 25 mcg/l;
sodium pyruvate present in a concentration from approximately 50 to approximately 150 mg/l;
D-biotin present in a concentration from approximately 1 to approximately 10 mg/l;
D-pantothenic acid present in a concentration from approximately 1 to approximately 15 mg/l;
folic acid present in a concentration from approximately 1 to approximately 10 mg/l;
niacinamide present in a concentration from approximately 1 to approximately 10 mg/l;
pyridoxine present in a concentration from approximately 1 to approximately 10 mg/l;
riboflavin present in a concentration from approximately 2 to approximately 20 mg/l;
thiamine present in a concentration from approximately 1 to approximately 5 mg/l; and
vitamin B12 present in a concentration from approximately 1 to approximately 10 mcg/l.

51. The method of claim 13, wherein the cell culture media further comprises:
L-Glutamine present in a concentration of approximately 2 mM/l;
L-Histidine present in a concentration of approximately 2 mg/l;
L-Methionine present in a concentration of approximately 1 mg/l;
L-Phenylalanine present in a concentration of approximately 2 mg/l;
L-Tryptophan present in a concentration of approximately 1 mg/l;
L-Tyrosine present in a concentration of approximately 2 mg/l;
L-Isoleucine present in a concentration of approximately 4 mg/l;
penicillin present in a concentration of approximately 100,000 IU/l;
streptomycin present in a concentration of approximately 100 mcg/l;
amphotericin B present in a concentration of approximately 2.5 mcg/l;
sodium heparin present in a concentration of approximately 10,000 IU/l;
choleric toxin present in a concentration of approximately 0.1 mg/l;
glucagon present in a concentration of approximately 1 mg/l;
hydrocortisone present in a concentration of approximately 10 mg/l;
recombinant human insulin present in a concentration of approximately 100 IU/l;
levothyroxine present in a concentration of approximately 50 mcg/l;
sodium bicarbonate present in a concentration of approximately 1.2 g/l;
sodium selenite present in a concentration of approximately 10 mg/l;
adenosine triphosphate present in a concentration of approximately 1 mg/l;
choline present in a concentration of approximately 1 mg/l;
cyticholine (histidine-5′-choline disphosphate) present in a concentration of approximately 10 mg/l;
ethanolamine present in a concentration of approximately 10 mg/l;
linoleic acid present in a concentration of approximately 10 mcg/l;
myo-inositol present in a concentration of approximately 1 mg/l;
oleic acid present in a concentration of approximately 10 mcg/l;
para-amino benzoic acid present in a concentration of approximately 1 mg/l;
phosphoethanolamine present in a concentration of approximately 5 mcg/l;
sodium pyruvate present in a concentration of approximately 50 mg/l;
D-biotin present in a concentration of approximately 1 mg/l;
D-pantothenic acid present in a concentration of approximately 1 mg/l;
folic acid present in a concentration of approximately 1 mg/l;
niacinamide present in a concentration of approximately 1 mg/l;
pyridoxine present in a concentration of approximately 1 mg/l;
riboflavin present in a concentration of approximately 2 mg/l;
thiamine present in a concentration of approximately 1 mg/l; and
vitamin B12 present in a concentration of approximately 1 mcg/l.

52. A method for biological tissue repair in a recipient, comprising the steps of:
extracting an Extremely Platelet Rich Plasma (EPRP) from whole blood;
activating coagulation of the Extremely Platelet Rich Plasma (EPRP) to form a Biological Glue; and
placing at least a portion of the Biological Glue at a biological site of the intended recipient to adhere at least one material at the biological site of the intended implant recipient.

53. A method for biological tissue repair in a recipient, comprising the steps of:
extracting an Extremely Platelet Rich Plasma (EPRP) from whole blood;
adding a material to the Extremely Platelet Rich Plasma (EPRP) to substantially disperse the material throughout the Extremely Platelet Rich Plasma (EPRP);
activating coagulation of the Extremely Platelet Rich Plasma (EPRP) to form a Biological Implant; and
placing at least a portion of the Biological Implant at a biological site of the intended recipient to at least partially occupy a space.
